(19) United States
(12) Patent Application Publication
(10) Pub. No.: US 2026/0117134 A1
(43) Pub. Date: Apr. 30, 2026

(54) SYSTEM AND METHOD FOR PREPARING HYDROGEN-RICH SYNGAS BY TAKING BIOGAS AS COMPOSITE GASIFICATION AGENT FOR GASIFICATION OF BIOGAS RESIDUE

(71) Applicant: Tianjin University, Tianjin (CN)

(72) Inventors: Guanyi CHEN, Tianjin (CN); Xiyue SUN, Tianjin (CN); Xiaochao ZHU, Tianjin (CN); Beibei YAN, Tianjin (CN); Zhi WANG, Tianjin (CN); Shengquan ZHOU, Tianjin (CN); Kaidi YANG, Tianjin (CN)

(73) Assignee: Tianjin University, Tianjin (CN)

(21) Appl. No.: 19/063,381

(22) Filed: Feb. 26, 2025

(30) Foreign Application Priority Data
Oct. 30, 2024 (CN) 202411524562.6

(51) Int. Cl. C10J 3/00; C12M 1/00; C12M 1/107

(52) U.S. Cl. CPC C10J 3/00; C12M 21/04; C12M 43/04; C12M 47/02; C12M 47/14; C10J 2300/0909; C10J 2300/0923; C10J 2300/0953; C10J 2300/0989; C10J 2300/1603; C10J 2300/1681

(57) ABSTRACT

A system and a method for preparing hydrogen-rich syngas by taking biogas as a composite gasification agent for the gasification of biogas residue are provided. The system includes an anaerobic fermenter for biomass gradient, a solid-liquid separation device, a drying reactor, a gasification reactor, a condensing tower, and a syngas purifier; the anaerobic fermenter for biomass gradient is connected to the drying reactor through the solid-liquid separation device, to transport separated biogas residue to the drying reactor, and the drying reactor and the anaerobic fermenter for biomass gradient are connected to the gasification reactor, respectively, to jointly input biogas and a biogas residue dried into the gasification reactor for reaction; and the gasification reactor is connected to the condensing tower, to transport reaction products to the condensing tower, the condensing tower is connected with the syngas purifier, performing purification treatment on a gas cooled, to obtain the hydrogen-rich syngas.

### CROSS REFERENCE TO THE RELATED APPLICATIONS

This application is based upon and claims priority to Chinese Patent Application No. 202411524562.6, filed on Oct. 30, 2024, the entire contents of which are incorporated herein by reference.

### TECHNICAL FIELD

The present disclosure relates to the technical field of treatment of solid wastes, and in particular, to a system and a method for preparing hydrogen-rich syngas by taking biogas as a composite gasification agent for gasification of biogas residue.

### BACKGROUND

With the continuous growth of global energy demand and the improvement of environmental protection awareness, the development and utilization of biomass resources has gradually become an important way to achieve circular agriculture and a low-carbon economy, and also an important support to achieve the goal of carbon neutrality. As a by-product of the fermentation process, biogas residue contains a certain amount of organic matter and nutrient elements, therefore, it is regarded as a potential biomass resource and has a large volume in China. However, at present, the treatment of biogas residue in China has the following problems: (1) High moisture content and complex composition: the complex structure and composition can bring challenges for the direct utilization of biogas residue. (2) High risk of environmental pollution: due to untreated biogas residue or mishandling, biogas residue can become a source of pathogens and nutrients, causing serious pollution to water and soil. (3) Low resource utilization rate: most biogas residues are still abandoned because they have not been effectively utilized.

Therefore, in order to avoid the above problems in the process of biogas residue resource utilization, to make full use of the organic matter in biogas residue, thereby improving the energy utilization efficiency of biogas residue after gasification, gasification technology is an efficient and safe biomass conversion technology, which can efficiently convert biogas residue into valuable syngas products. Nevertheless, biomass gasification technology is affected by many factors, wherein the type of gasification agents has a crucial influence on the chemical reactions during the biomass gasification process.

Commonly used gasification agents mainly include air, oxygen, carbon dioxide and mixed gasification agents. Due to the difference in the content of functional elements such as C, H, and so forth, different gasification agents affect the composition of key combustible gas components (CH4, H2, CO, etc.) in syngas, which in turn affects the calorific value of the product gas. However, the higher nitrogen content in the air leads to the lower calorific value of the syngas, and the higher oxygen content makes the degree of gasification difficult to control. Oxygen gasification can reduce the dilution of nitrogen to a certain extent and increase the calorific value of syngas, but the gas products are difficult to have hydrogen-rich characteristics. Carbon dioxide gasification requires additional steps for capturing and purifying carbon dioxide, thus increasing costs. Therefore, the currently developed gasification technology for biogas residue cannot convert the gasification of biogas residue into high-quality hydrogen-rich and carbon monoxide-rich syngas more efficiently. Especially, at present, the urgent demand for clean fuels such as green methanol and aviation kerosene requires biomass gasification syngas must possess high hydrogen-rich characteristics. Therefore, it is crucial to develop a low-cost and easy-to-obtain system and method for preparing hydrogen-rich syngas by taking biogas as a composite gasification agent for gasification of biogas residue.

### SUMMARY

In order to solve at least one technical problem in the background, the present disclosure provides a system and a method for preparing hydrogen-rich syngas by taking biogas as a composite gasification agent for the gasification of biogas residue, which can utilize biogas as the composite gasification agent for gasification of biogas residue, thereby achieving a significant increase in the hydrogen yield.

In order to achieve the above effects, the present disclosure provides a system for preparing hydrogen-rich syngas by taking biogas as a composite gasification agent for gasification of biogas residue, wherein the system includes an anaerobic fermenter for biomass gradient, a solid-liquid separation device, a drying reactor, a gasification reactor, a condensing tower, a syngas purifier; and

the anaerobic fermenter for biomass gradient is connected to the drying reactor through the solid-liquid separation device, to transport separated biogas residue to the drying reactor, and the drying reactor and the anaerobic fermenter for biomass gradient are connected to the gasification reactor, respectively, to jointly input biogas produced by the anaerobic fermenter for biomass gradient and a biogas residue dried by the drying reactor into the gasification reactor for reaction; and the gasification reactor is connected to the condensing tower, to transport reaction products to the condensing tower, the condensing tower is connected with the syngas purifier, performing purification treatment on a gas cooled by the condensing tower through the syngas purifier, to obtain the hydrogen-rich syngas.

In some embodiments, the system further includes a biogas collector and a biogas storage device, and the anaerobic fermenter for biomass gradient is connected to the gasification reactor through the biogas collector and the biogas storage device in turn.

In some embodiments, the system further includes a liquid waste collection device and a water resource treatment device, the solid-liquid separation device is connected to the liquid waste collection device and the water resource treatment device in turn, to collect and purify separated biogas slurry.

A method for preparing hydrogen-rich syngas by taking biogas as a composite gasification agent for gasification of biogas residue includes the following steps:
step S10, producing biogas by an anaerobic fermenter for biomass gradient, and transporting the produced biogas to a gasification reactor;
step S20, performing a drying process through a drying reactor on a biogas residue separated by a solid-liquid separation device, then transporting a dried biogas residue to the gasification reactor; and
step S30, transporting reaction products obtained by the gasification reactor to the condensing tower, performing purification treatment on a gas cooled by the condensing tower through a syngas purifier, to obtain the hydrogen-rich syngas.

In some embodiments, in the step S10, the biogas produced by the anaerobic fermenter for biomass gradient is collected into a biogas storage device by a biogas collector, and a proportion of biogas and nitrogen in the gasification reactor is controlled.

In some embodiments, the method further includes step S40, performing collection and purification on a biogas slurry separated by the solid-liquid separation device through a liquid waste collection device and a water resource treatment device.

The beneficial effects of the present disclosure are:

Aiming at biogas residue, a typical biomass feedstock that is difficult to achieve resource utilization, in order to realize high-efficiency clean utilization of biogas residue, the present disclosure provides a system and a method for preparing hydrogen-rich syngas by taking biogas as a composite gasification agent for gasification of biogas residue, which can utilize biogas as the composite gasification agent for gasification of biogas residue, thereby achieving a significant increase in hydrogen yield, and further producing hydrogen-rich syngas through consuming energy on-site of fermented biogas residue, which improves the quality of gas products and achieves high-efficiency and clean thermo-chemical conversion of biomass biogas residue, thereby providing a new idea and technical reference for the industrial application of biogas residue biomass.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flowchart of a method of the present disclosure;

FIGS. 2A-2B show distribution schematic diagrams of gas products under different atmosphere conditions of the present disclosure;

FIGS. 3A-3B show schematic diagrams yields for H₂/CO and H₂ under different atmosphere conditions of the present disclosure;

FIG. 4 shows a schematic diagram of cold gas efficiency of gasification of biogas residue under different atmosphere conditions of the present disclosure;

FIGS. 5A-5B show distribution schematic diagrams of three-phase products in biogas residue gasification under different atmosphere conditions of the present disclosure; and

FIG. 6 shows a schematic diagram of raw material consumption for hydrogen production.

### DETAILED DESCRIPTION OF THE EMBODIMENTS

In the following, the technical solutions in the embodiments of the present disclosure will be clearly and completely described with reference to the drawings in the embodiments of the present disclosure. Obviously, the described embodiments are only a part of the embodiments of the present disclosure, but not all the embodiments thereof. Based on the embodiments of the present disclosure, all other embodiments obtained by those skilled in the art without any creative efforts shall fall within the scope of the present disclosure.

It is important to note that in the specification, claims, and the above drawings of the present disclosure, terms such as ‘first’ and ‘second’ are used only to distinguish similar objects, and do not necessarily require or imply the order or precedence order. It is to be understood that the data used in this way can be interchanged in appropriate cases for the embodiments described herein. In addition, the terms ‘include’, ‘possess’ and any other variation thereof are intended to cover a non-exclusive inclusion. For example, a process, method, system, product, or device that contains a series of steps or units need not be limited to those steps or units that are clearly listed but may include other steps or units that are not clearly listed or are inherent to those processes, methods, products, or devices.

In the present disclosure, orientation or positional relationships indicated by terms ‘up’, ‘down’, ‘left’, ‘right’, ‘front’, ‘back’, ‘to’, ‘bottom’, ‘inside’, ‘outside’, ‘middle’, ‘vertical’, ‘horizontal’, ‘transverse’, ‘longitudinal’, and so forth are the orientation or positional relationships shown based on the drawings. These terms are primarily intended to better describe the present application and embodiments thereof, but are not intended to limit that the indicated device, component, or component must have a specific orientation, or be constructed and operated in a specific orientation.

Moreover, some of the above terms may be used to express other meanings in addition to the orientation or positional relationship. For example, the term ‘up’ may also be used to represent a certain attachment or connection relationship in some cases. For those skilled in the art, the specific meaning of these terms in the present application can be understood according to the specific situation.

Besides, the terms ‘install’, ‘arrange’, ‘provided with’, ‘connect’, ‘joined by’, and ‘sleeve connected’ should be understood in a broad sense. For example, it may be a fixed connection, a detachable connection, or an integral structure; it may be a mechanical connection or an electrical connection; and it may be directly connected, or indirectly connected through an intermediate medium, or an internal connection between two devices, components or components. For those skilled in the art, the specific meaning of the above terms in the present application can be understood according to the specific situation.

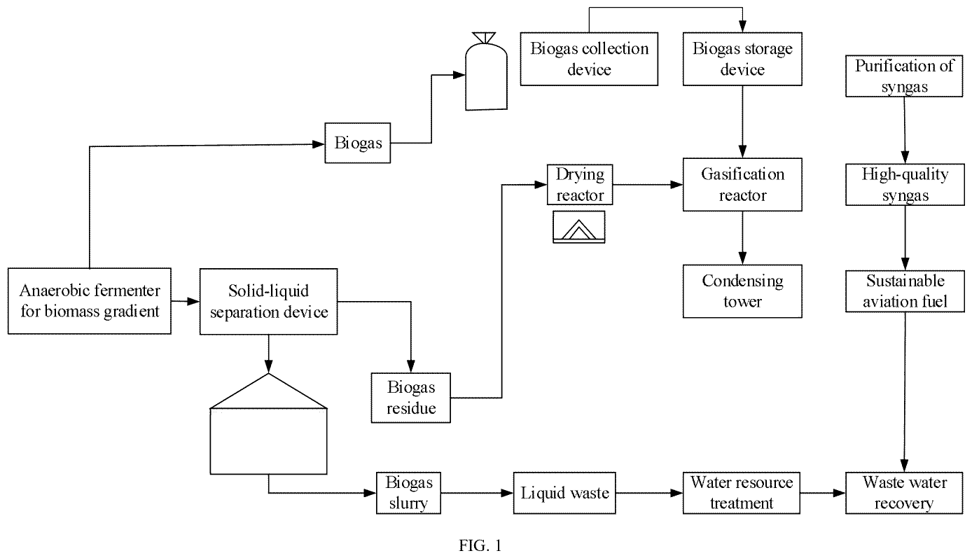

In order to achieve the above effects, as shown in FIG. 1, the present disclosure provides a system for preparing hydrogen-rich syngas by taking biogas as a composite gasification agent for gasification of biogas residue, wherein the system includes an anaerobic fermenter for biomass gradient, a solid-liquid separation device, a drying reactor, a gasification reactor, a condensing tower, a syngas purifier; and

the anaerobic fermenter for biomass gradient is connected to the drying reactor through the solid-liquid separation device, to transport separated biogas residue to the drying reactor, and the drying reactor and the anaerobic fermenter for biomass gradient are connected to the gasification reactor, respectively, to jointly input biogas produced by the anaerobic fermenter for biomass gradient and a biogas residue dried by the drying reactor into the gasification reactor for reaction; and the gasification reactor is connected to the condensing tower, to transport reaction products to the condensing tower, the condensing tower is connected with the syngas purifier, performing purification treatment on a gas cooled by the condensing tower through the syngas purifier, to obtain the hydrogen-rich syngas.

In some embodiments, the system further includes a biogas collector and a biogas storage device, and the anaerobic fermenter for biomass gradient is connected to the gasification reactor through the biogas collector and the biogas storage device in turn.

In some embodiments, the system further includes a liquid waste collection device and a water resource treatment device, the solid-liquid separation device is connected to the liquid waste collection device and the water resource treatment device in turn, to collect and purify separated biogas slurry.

The main components of biogas are CH₄, CO₂, and so forth, wherein as a hydrogen donor in the methanation reaction of gasification reaction, CH₄ can enhance the effect of hydrogen production by biomass gasification; meanwhile, as a gasification agent, CO₂ can promote the Boudouard reaction to a certain extent and significantly increase CO production, thereby providing a higher reaction temperature, faster reaction rate, larger product range, and higher thermal efficiency at the same molar ratio, and being able to achieve a higher synthesis gas calorific value. Jointly as products of anaerobic fermentation, biogas and biogas residue realize the in-situ utilization of biogas by means of gasification, which cannot only save energy consumption and losses during the transportation of biogas, achieve on-site consumption, but also strengthen the energy utilization of biogas residue. Therefore, biogas can be used as a superior gasification agent for realizing the high-value conversion of biogas residue gasification.

In order to solve the problem of low efficiency in the treatment and energy utilization of biogas residue, the present disclosure utilizes part of biogas in biogas project to achieve resource treatment of biogas residue, while improving the conversion rate of biomass in the entire system to high-quality hydrogen-rich synthesis gas. Traditional gasification methods, such as pure nitrogen gasification and CO₂ gasification, have the disadvantages of low hydrogen yield and inefficient energy efficiency. Therefore, the present disclosure provides a a system for preparing hydrogen-rich syngas by taking biogas as a composite gasification agent for gasification of biogas residue. The system can effectively improve the gasification efficiency of biogas residue and the production rates of H₂ and CO, and ensure the safety of the experiment. The present disclosure has the characteristics of high efficiency, cost-saving and environmental friendliness, so it has good market application value.

The present disclosure further provides a method for preparing hydrogen-rich syngas by taking biogas as a composite gasification agent for gasification of biogas residue, including the following steps:
step S10, biogas is produced by an anaerobic fermenter for biomass gradient, and the produced biogas is transported to a gasification reactor;
step S20, a drying process is performed on a biogas residue separated by a solid-liquid separation device through a drying reactor, and then a dried biogas residue is transported to the gasification reactor; and
step S30, reaction products obtained from the gasification reactor are transported to a condensing tower, and purification treatment is performed on a gas cooled by the condensing tower through a syngas purifier, to obtain the hydrogen-rich syngas.

In some embodiments, in the step S10, the biogas produced by the anaerobic fermenter for biomass gradient is collected into a biogas storage device by a biogas collector, and a proportion of biogas and nitrogen in the gasification reactor is controlled.

In some embodiments, the method further includes step S40, wherein collection and purification are performed on a biogas slurry separated by the solid-liquid separation device through a liquid waste collection device and a water resource treatment device.

The present disclosure has the following advantages:
Utilizing the biogas as a composite gasification agent: the present disclosure proposes to take biogas as the compound gasification agent in the gasification process, compared with traditional gasification agents like air, oxygen or water vapor, the introduction of biogas not only effectively utilizes resources and reduces exhaust emissions, but also significantly improves the hydrogen yield in the gasification process through its high methane content. In addition, the use of biogas can reduce the dependence on external gasification agents, thereby achieving more economical operation. According to the present disclosure, it is found that the hydrogen yield under biogas gasification is increased by 50.8% compared to that under pure nitrogen gasification, and is increased by 40.3% compared to that under CO₂ atmosphere. Moreover, the H₂/CO ratio of syngas produced under the biogas atmosphere is 1.55 to 1.96 times higher than that of CO₂ gasification.

Increasing hydrogen production: the gasification of biogas residue usually limits hydrogen production due to a low carbon-hydrogen ratio, while the present disclosure takes the methane content in the biogas as an additional hydrogen source, which not only promotes the deep gasification of biogas residue, but also enhances the production of hydrogen, thereby generating hydrogen-rich syngas and improving the application potential of this technology in hydrogen energy production.

Integrated and high-efficiency gasification process design in the system: the biogas residue-biogas gasification system developed by the present disclosure has significant advantages in energy utilization efficiency. The system design fully considers the supply and control of biogas, optimizes the gasification reaction conditions, and ensures the efficiency and stability of the gasification reaction. Through the highly efficient reactor structure design and heat exchange technology, the system can realize the self-circulation of energy, improve overall efficiency and reduce energy consumption. The results show that the cold gas efficiency under a biogas atmosphere is 2.39 to 3.06 times higher than that under CO₂ gasification.

Optimization of gasification products of biogas residue: the biogas gasification system of the present disclosure has a significant effect on the gas composition and distribution of three-phase products in the gasification products of biogas residue. The system design can optimize the gas composition, increase the production rate of useful gas, and improve the distribution characteristics of three-phase products.

Collaborative optimization of gas purification and hydrogen-rich syngas preparation: the method not only focuses on the generation of hydrogen during gasification, but also significantly reduces the content of impurity gases such as carbon dioxide and carbon monoxide through subsequent gas purification technology, thereby improving the quality of synthesis gas, and making it more suitable for downstream industrial applications, such as chemical synthesis and the preparation of sustainable aviation fuel.

In the following, the significant advantages of biogas gasification of biogas residue are explained in detail.

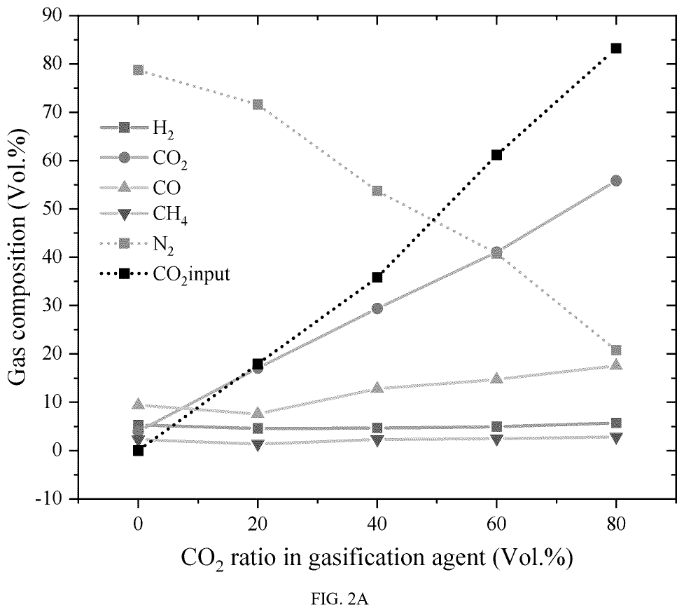

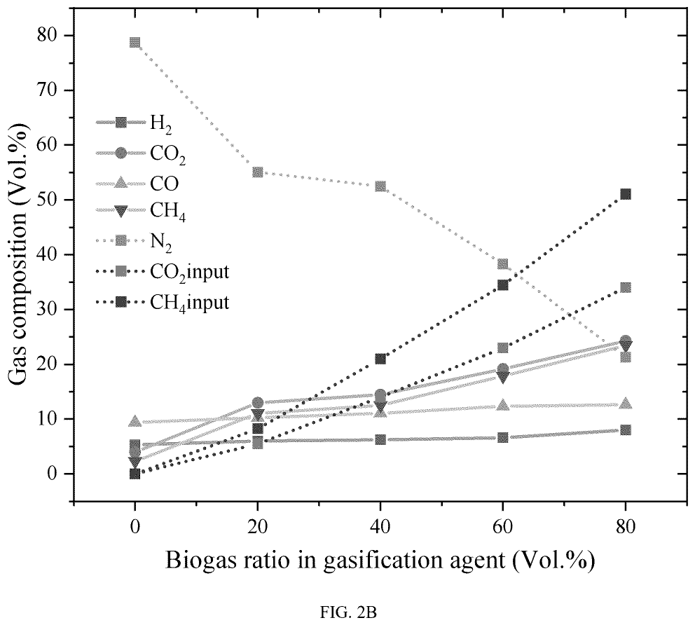

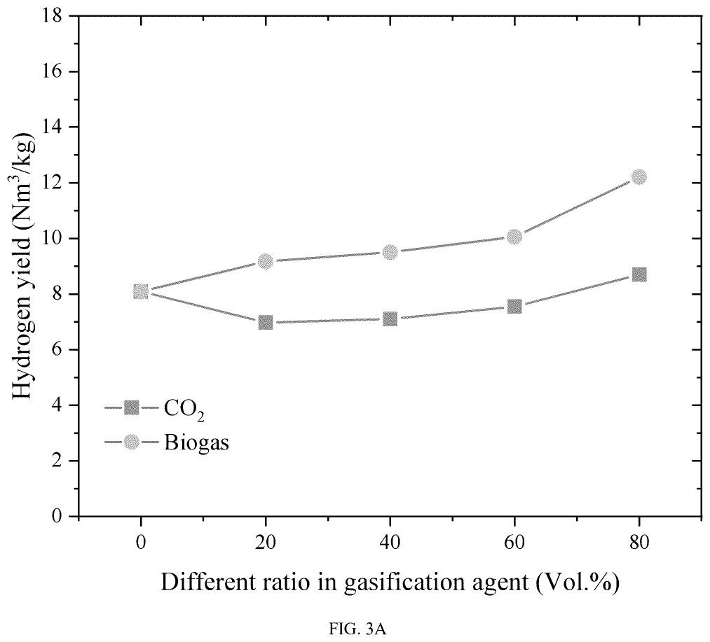

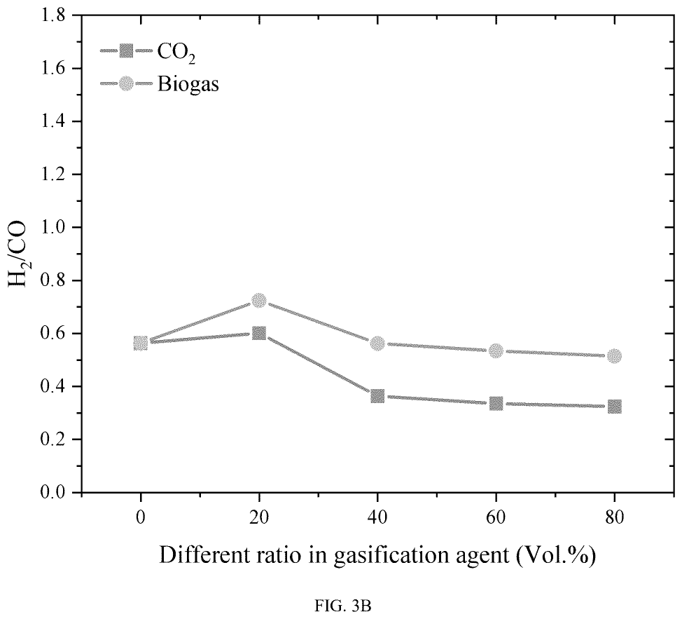

1. Under the biogas atmosphere, the thermal properties of the product gas can be improved, so that the content of H₂ and CO can be significantly increased, with the characteristics of improving the gas quality. As shown in FIG. 2A, FIG. 2B, FIG. 3A, and FIG. 3B. The proportion of combustible components (such as H₂ and CO) in syngas is higher, with a high fuel value. High purity of syngas: the syngas has fewer impurities, which enhances its application value in subsequent processes, like fuel cells or chemical synthesis.

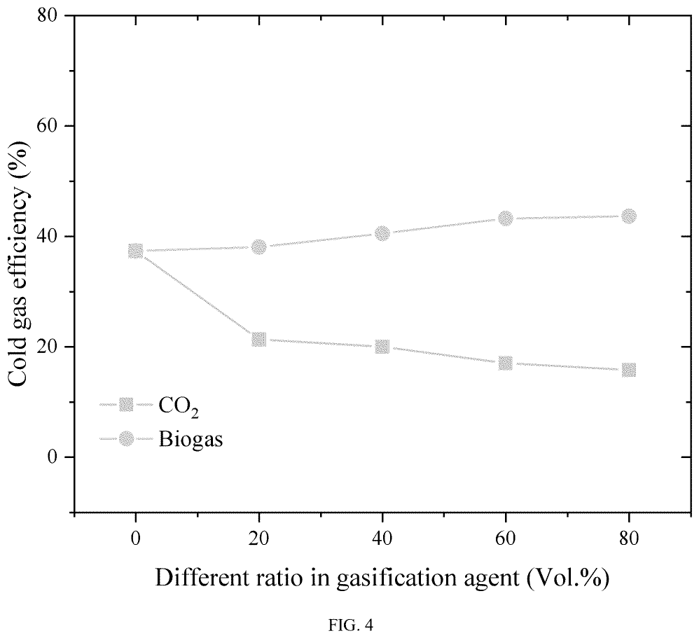

2. The biogas gasification of biogas residue has significant advantages in energy utilization efficiency. It can be seen from FIG. 4 that the cold gas efficiency in biogas gasification is significantly higher than that in CO₂ gasification, so that more raw material energy is successfully converted into chemical energy in syngas rather than being wasted. This shows that the smaller the energy loss in the reaction process is, the less the heat loss and loss of unreacted carbon in the gasification process are. The reaction is more efficient, and the reaction conditions (such as temperature, pressure, and gasification agent ratio) are well optimized, which promotes the complete gasification and high-efficiency conversion of raw materials.

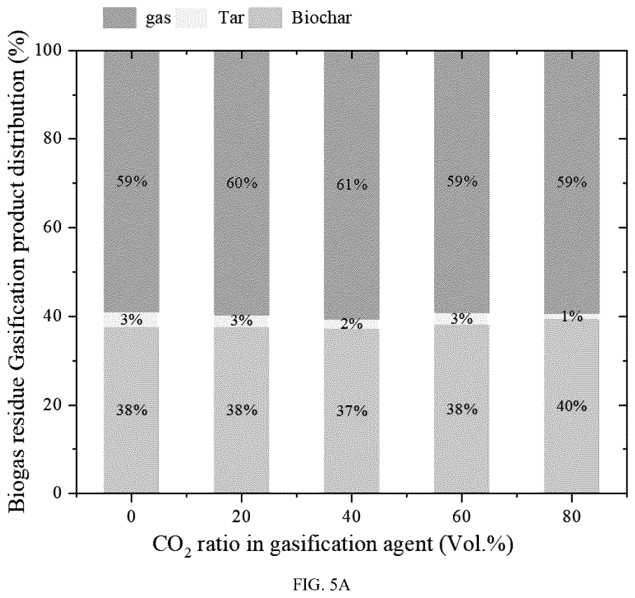

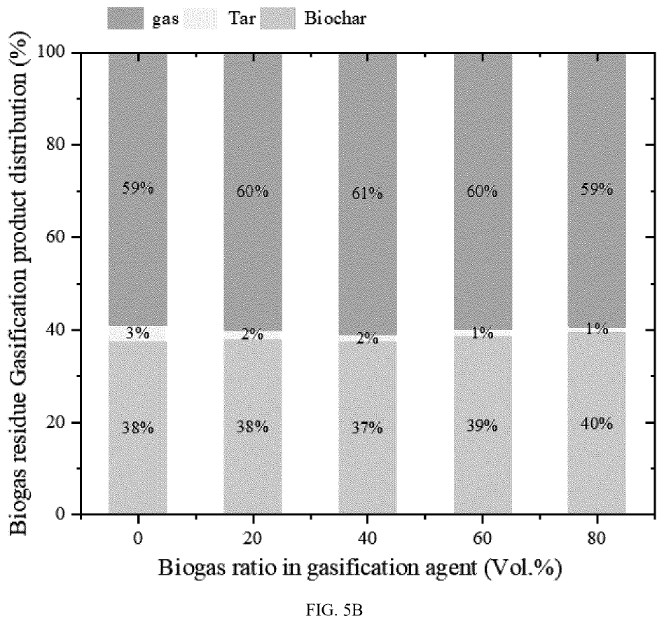

3. The gasification products of biogas residue in the biogas atmosphere have significant advantages. The production of gas products in the gasification of biogas residue which is stable between 59% and 61% and the proportion of gas products under CO₂ atmosphere are shown in FIG. 5A and FIG. 5B, wherein the performance of gas produced under the biogas atmosphere is higher. In addition, in the gasification of biogas residue, the production of tar oil is lower, being decreased from 3% to 1%. The low production of tar oil means that the complex organic compounds to be treated in the gasification process are reduced, which contributes to improving the operational stability and efficiency of the system. Because CO₂ in biogas can promote the partial oxidation of carbon in the gasification process to produce carbon monoxide, at the same time, it avoids the generation of too much tar oil. The production of biochar is between 38% and 40%, which fluctuates slightly but remains stable. This shows that the gasification process of biogas has little effect on the formation of solid carbon, and can provide stable biochar output, which is convenient for further treatment and utilization. Because the partial oxidation of carbon and the reforming reaction of methane are balanced under the biogas atmosphere, so that the generation of biochar is more stable. This characteristic contributes to providing a sustained output of solid products, facilitating further utilization of carbon-based materials, such as soil improvement or carbon capture.

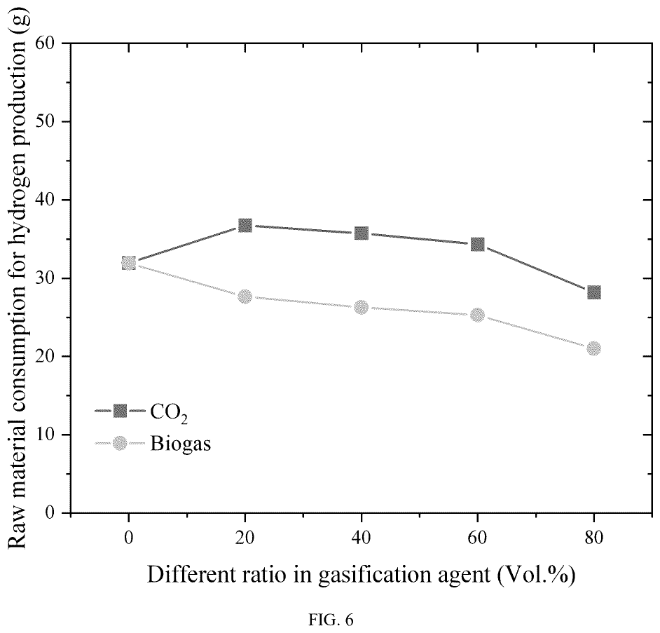

4. When the same 1 g of hydrogen is produced, less raw material is consumed under the condition of the biogas atmosphere, as shown in FIG. 6. When producing products of the same quality, a close relationship exists between the amount of raw material consumption and environmental benefits. Less raw material consumption usually means higher resource efficiency. It not only reduces the exploitation and consumption of natural resources, but also reduces the environmental impact related to the acquisition of raw materials, so it has positive environmental benefits. At the same time, it can reduce the energy consumption required for raw material extraction and fermentation processes, thereby reducing greenhouse gas emissions and other pollutant emissions. Reducing the consumption of raw materials can lower the environmental impact throughout the entire production cycle, including global warming potential, acidification potential, and eutrophication potential. Therefore, reducing raw material consumption when producing products of the same quality can bring significant environmental benefits, which not only contributes to protecting natural resources and the ecological environment, but also conforms to the goal of sustainable development and circular economy. Reducing raw material consumption has achieved the effect of further reducing the cost of the production stage, thereby improving economic efficiency.

The above descriptions are only preferred embodiments of the present disclosure, rather than any limitation on the technical scope of the present disclosure. Therefore, any minor changes, equivalent variations and modifications to the above embodiments based on the technical substance of the present disclosure are all still within the scope of the technical solution of the present disclosure.

What is claimed is:

1. A system for preparing a hydrogen-rich syngas by taking a biogas as a composite gasification agent for a gasification of a biogas residue, comprising an anaerobic fermenter for a biomass gradient, a solid-liquid separation device, a drying reactor, a gasification reactor, a condensing tower, and a syngas purifier; and
wherein the anaerobic fermenter for the biomass gradient is connected to the drying reactor through the solid-liquid separation device, to transport a separated biogas residue to the drying reactor, the drying reactor and the anaerobic fermenter for the biomass gradient are connected to the gasification reactor, respectively, to jointly input the biogas produced by the anaerobic fermenter for the biomass gradient and a biogas residue dried by the drying reactor into the gasification reactor for a reaction; and the gasification reactor is connected to the condensing tower, to transport reaction products to the condensing tower, the condensing tower is connected with the syngas purifier, and a purification treatment is performed on a gas cooled by the condensing tower through the syngas purifier, to obtain the hydrogen-rich syngas.

2. The system for preparing the hydrogen-rich syngas by taking the biogas as the composite gasification agent for the gasification of the biogas residue according to claim 1, further comprising a biogas collector and a biogas storage device, wherein the anaerobic fermenter for the biomass gradient is connected to the gasification reactor through the biogas collector and the biogas storage device in turn.

3. The system for preparing the hydrogen-rich syngas by taking the biogas as the composite gasification agent for the gasification of the biogas residue according to claim 1, further comprising a liquid waste collection device and a water resource treatment device, wherein the solid-liquid separation device is connected to the liquid waste collection device and the water resource treatment device in turn, to collect and purify a separated biogas slurry.

4. A method for preparing a hydrogen-rich syngas by taking a biogas as a composite gasification agent for a gasification of a biogas residue, comprising the following steps:
step S10, producing the biogas by an anaerobic fermenter for a biomass gradient, and transporting a produced biogas to a gasification reactor;
step S20, performing a drying process through a drying reactor on the biogas residue separated by a solid-liquid separation device, and then transporting a dried biogas residue to the gasification reactor; and
step S30, transporting reaction products obtained by the gasification reactor to a condensing tower, and performing a purification treatment on a gas cooled by the condensing tower through a syngas purifier, to obtain the hydrogen-rich syngas.

5. The method for preparing the hydrogen-rich syngas by taking the biogas as the composite gasification agent for the gasification of the biogas residue according to claim 4, wherein in the step S10, the produced biogas from the anaerobic fermenter for the biomass gradient is collected into a biogas storage device by a biogas collector, and a proportion of the biogas and nitrogen in the gasification reactor is controlled.

6. The method for preparing the hydrogen-rich syngas by taking the biogas as the composite gasification agent for the gasification of the biogas residue according to claim 4, further comprising: step S40, performing a collection and a purification on a biogas slurry separated by the solid-liquid separation device through a liquid waste collection device and a water resource treatment device.

7. The system for preparing the hydrogen-rich syngas by taking the biogas as the composite gasification agent for the gasification of the biogas residue according to claim 2, further comprising a liquid waste collection device and a water resource treatment device, wherein the solid-liquid separation device is connected to the liquid waste collection device and the water resource treatment device in turn, to collect and purify a separated biogas slurry.

8. The method for preparing the hydrogen-rich syngas by taking the biogas as the composite gasification agent for the gasification of the biogas residue according to claim 5, further comprising: step S40, performing a collection and a purification on a biogas slurry separated by the solid-liquid separation device through a liquid waste collection device and a water resource treatment device.
